(19) United States
(12) Patent Application Publication
(10) Pub. No.: US 2023/0025182 A1
(43) Pub. Date: Jan. 26, 2023

(54) SYSTEM AND METHODS FOR ULTRASOUND ACQUISITION WITH ADAPTIVE TRANSMITS

(71) Applicant: GE Precision Healthcare LLC, Milwaukee, WI (US)

(72) Inventors: Rahul Venkataramani, Bangalore (IN); Vikram Melapudi, Bangalore (IN)

(21) Appl. No.: 17/381,113

(22) Filed: Jul. 20, 2021

(51) Int. Cl. G06T 7/00; A61B 8/00

(52) U.S. Cl. CPC G06T 7/0014; A61B 8/54; A61B 8/463; G06T 2207/10132; G06T 2207/20084; G06T 2207/20004; G06T 2207/20081

(57) ABSTRACT

Methods and systems are provided for dynamically selecting ultrasound transmits. In one example, a method includes dynamically updating a number of transmit lines and/or a pattern of transmit lines for acquiring an ultrasound image based on a prior ultrasound image and a task to be performed with the ultrasound image, and acquiring the ultrasound image with an ultrasound probe controlled to operate with the updated number of transmit lines and/or the updated pattern of transmit lines.

### TECHNICAL FIELD

Embodiments of the subject matter disclosed herein relate to ultrasound imaging, and more particularly, to improving image quality for ultrasound imaging.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In one embodiment, a method includes dynamically updating a number of transmit lines and/or a pattern of transmit lines for acquiring an ultrasound image based on a prior ultrasound image and a task to be performed with the ultrasound image, and acquiring the ultrasound image with an ultrasound probe controlled to operate with the updated number of transmit lines and/or the updated pattern of transmit lines.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an ultrasound system;

FIGS. 2A-2C show sets of transmit lines of scan sequences that may be executed to acquire ultrasound information that may be used to generate ultrasound images;

FIG. 3 is a schematic diagram illustrating a system for acquiring ultrasound images at optimized transmit settings using an adaptive transmit model, according to an exemplary embodiment;

FIG. 4 schematically shows a reinforcement learning architecture for training an adaptive transmit model, according to an embodiment;

FIG. 5 is a flow chart illustrating an example method for selecting transmit parameters during ultrasound imaging using an adaptive transmit model, according to an exemplary embodiment;

FIG. 6 is a flow chart illustrating an example method for training an adaptive transmit model; and

FIGS. 7A-7C show example transmit lines for acquiring ultrasound images according to embodiments of the disclosure.

### DETAILED DESCRIPTION

Medical ultrasound imaging typically includes the placement of an ultrasound probe including one or more transducer elements onto an imaging subject, such as a patient, at the location of a target anatomical feature (e.g., abdomen, chest, etc.). Images are acquired by the ultrasound probe and are displayed on a display device in real time or near real time (e.g., the images are displayed once the images are generated and without intentional delay). The operator of the ultrasound probe may view the images and adjust various acquisition parameters and/or the position of the ultrasound probe in order to obtain high-quality images of the target anatomical feature (e.g., the heart, the liver, the kidney, or another anatomical feature). The acquisition parameters that may be adjusted include transmit parameters including the number and/or the pattern of transmit lines (also referred to as transmits). A transmit line may include a focused pulse of ultrasound at a given steering angle, generated by one or more ultrasound transducer elements. During imaging, a plurality of transmit lines at different steering angles may be produced to obtain the imaging data for forming an image. While increasing the number of transmit lines may improve image resolution, higher transmits lower the frame rate of the imaging. Thus, there is a balance between imaging with a sufficient number of transmits to acquire images of desired resolution while maintaining a reasonably fast frame rate. In particular, when imaging moving objects, such as the heart or lungs, faster frame rates may be desired to reduce motion-induced artifacts.

Thus, according to embodiments disclosed herein, an adaptive transmit model may be trained using reinforcement learning techniques to adaptively select an optimal pattern and number of transmits for an acquisition of an ultrasound image depending on an image being acquired and a task for which the acquisition is performed (e.g., detecting B-lines in a lung imaging scan). By training the adaptive transmit model with reinforcement learning techniques, a reward may be calculated during training such that the adaptive transmit model may seek configurations of transmit lines during ultrasound scans that balance image resolution and frame rate in a manner best suited for a particular imaging or diagnostic task according to the reward.

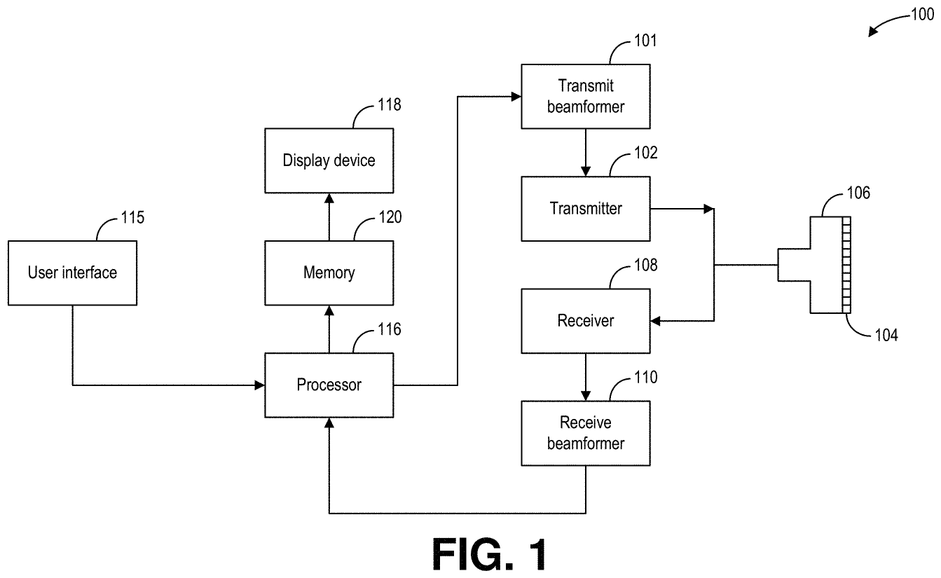

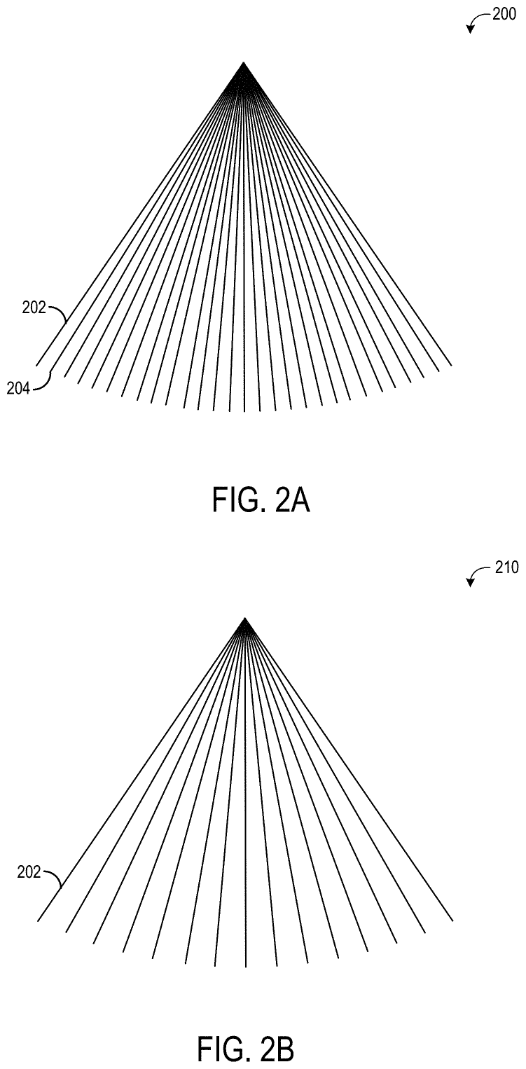

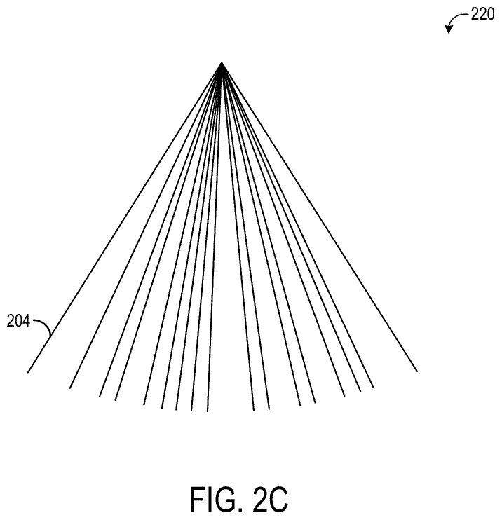

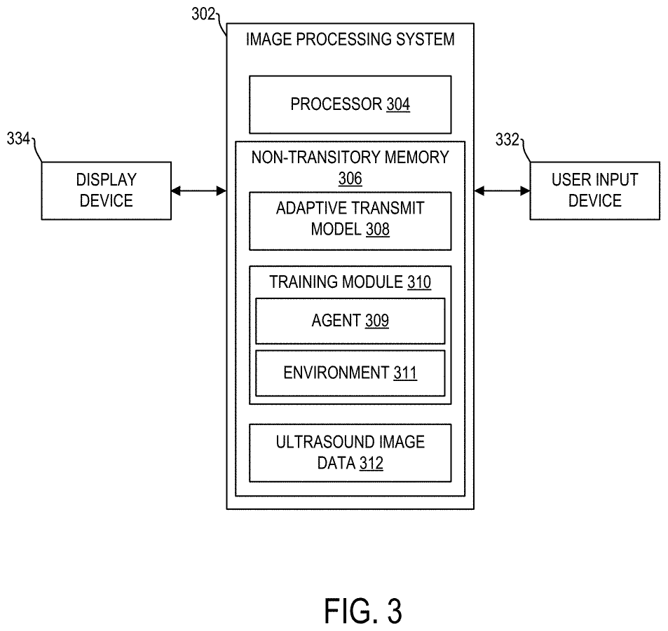

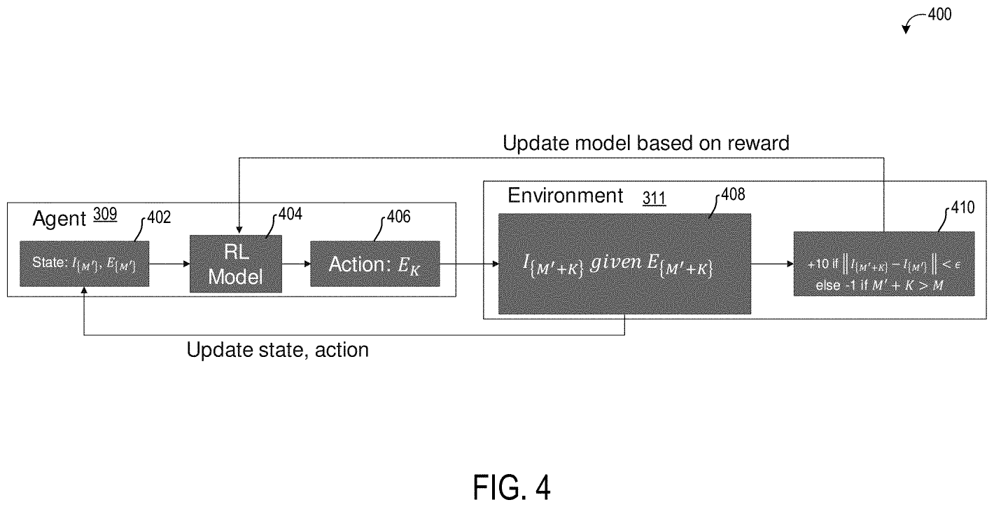

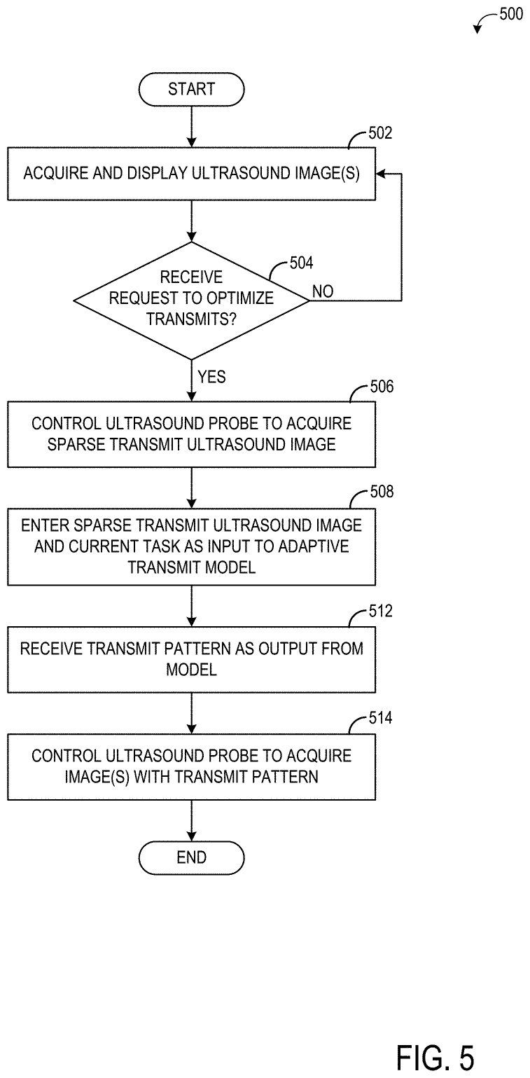

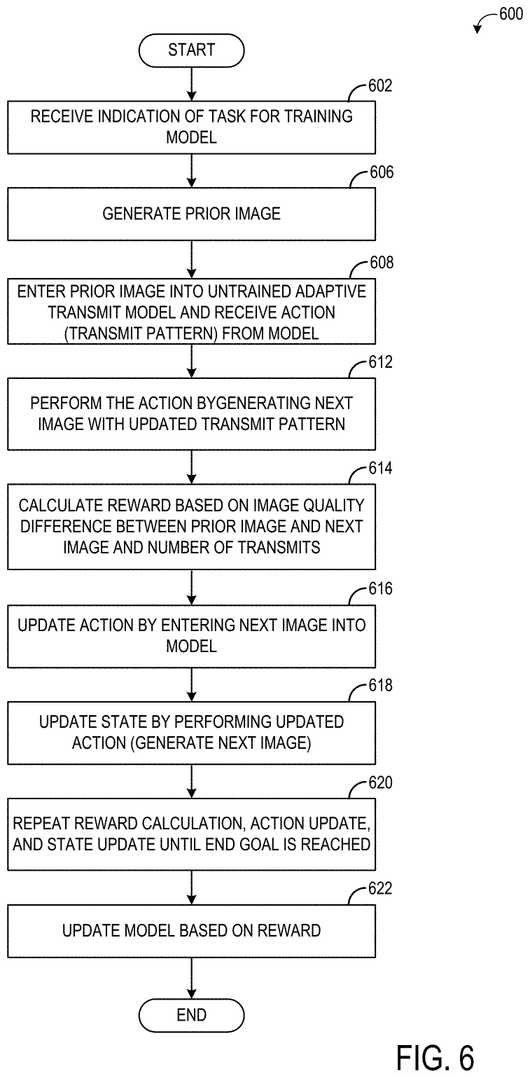

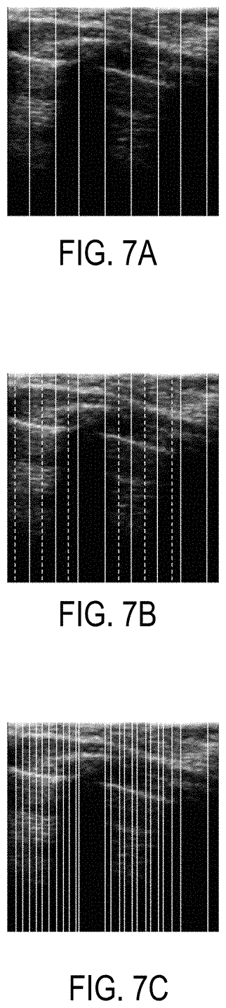

An example ultrasound system including an ultrasound probe, a display device, and an imaging processing system are shown in FIG. 1. Via the ultrasound probe, ultrasound images may be acquired and displayed on the display device. Patterns of high-frequency pulses (transmit lines) are shown in FIGS. 2A-2C. An image processing system, as shown in FIG. 3, includes an adaptive transmit model which may be trained according to a reinforcement learning architecture depicted in FIG. 4, to select a transmit line pattern during an ultrasound imaging scan to achieve a goal frame rate, resolution, and the like. The adaptive transmit model may be deployed according to the method of FIG. 5 and trained according to the method of FIG. 6 in order to configure optimal transmit line patterns. As shown in FIGS. 7A-7C, the adaptive transmit model may configure a transmit line pattern for a specific imaging task.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment.

According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a CPU, a digital signal processor, a field-programmable gate array, and a graphic board. In some examples, the processor 116 may also include a complex demodulator (not shown) that demodulates the RF data and generates raw data. In another embodiment, the demodulation can be carried out earlier in the processing chain.

The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

Ultrasound images acquired by ultrasound imaging system 100 may be further processed. In some embodiments, ultrasound images produced by ultrasound imaging system 100 may be transmitted to an image processing system, where in some embodiments, the ultrasound images may be analyzed by one or more machine learning models trained using a reinforcement learning mechanism in order to determine optimal transmit patterns for acquiring ultrasound images for a given task and anatomy.

Although described herein as separate systems, it will be appreciated that in some embodiments, ultrasound imaging system 100 includes an image processing system. In other embodiments, ultrasound imaging system 100 and the image processing system may comprise separate devices. In some embodiments, images produced by ultrasound imaging system 100 may be used as a training data set for training one or more machine learning models, wherein the machine learning models may be used to perform one or more steps of ultrasound image processing, as described below.

FIGS. 2A-2C show sets of transmit lines in example patterns. FIG. 2A shows an example set of transmit lines 200 of a first example scan sequence that may be executed to acquire ultrasound information that may be used to generate a single image. Each line in the set of transmit lines 200, such as first transmit line 202 and second transmit line 204, represents a transmit direction, and the transmits are fired sequentially from, for example, left to right. The set of transmit lines 200 may represent a maximum number of transmits possible for an ultrasound probe. For each transmit line in the set of transmit lines 200, the ultrasound probe may send one or more pulses of sound in a direction of the transmit line (e.g., activating one or more transducer elements per transmit). The first example scan sequence includes the highest number of transmits that the ultrasound probe is capable of firing, and thus may result in lower image frame rates (e.g., relative to other scan sequences with fewer transmits), which may reduce a likelihood of discerning rapid movements (e.g., valve leaflets or fetus movements) across ultrasound images and/or increase motion-related image artifacts. The first example scan sequence may also transmit in regions that do not improve image clarity, as a result of a consistent uniform scanning pattern. For example, the first example scan sequence may transmit in regions that do not include anatomical features of interest.

FIG. 2B shows an example set of transmit lines 210 of a second example scan sequence that may be executed to acquire ultrasound information that may be used to generate a single image. The set of transmit lines 210 may represent a first fixed transmit pattern with a reduced number of equally spaced transmits, such that every other transmit (e.g., second transmit line 204) depicted in the set of transmit lines 200 of the first example scan sequence does not occur. While the second example scan sequence may result in a higher frame rate than the first example scan sequence, the second example scan sequence may generate images having lower image quality as a result of fewer transmits. Further, because the transmit pattern is fixed, the transmits may not be optimized for an imaging task and as a result excess image information in areas outside an area of interest (e.g., an anatomical feature that is the target of the scan) may be obtained. In this way, uniformly spaced transmits may not optimally transmit in a target area for an imaging task.

FIG. 2C shows an example set of transmit lines 220 of a third example scan sequence that may be executed to acquire ultrasound information that may be used to generate a single image. The set of transmit lines 220 may represent a second fixed transmit pattern with a reduced number of unevenly spaced transmits, such that the transmits may be spaced for a specific imaging task. In one example, the set of transmit lines 220 may represent a fixed transmit pattern for a lung imaging task to detect B-lines. The third example scan sequence may therefore be configured to direct transmits in a predicted area of interest based on the imaging task while minimizing transmits outside the area of interest. However, without taking into consideration the specifics of the image itself (e.g., where the area of interest is located in the image frame, particularities of the subject's anatomy), the third example scan sequence may instead target areas outside of the area of interest for the imaging task. Thus, each of the first, second, and third example scan sequences may result in sub-optimal imaging (e.g., low frame rate, low resolution, and/or acquisition of unnecessary image data).

In obtaining an ultrasound image with optimal image resolution and frame rate, a fixed number of transmit lines as well as a placement of transmit lines may accomplish high frame rate but at a cost of a lower image resolution or accomplish high image resolution but at a cost of a lower frame rate. Transmit selections that are based on specific regions (e.g., lungs, liver) may still not configure optimal transmit patterns as a result of abnormally sized or shaped regions of interest or other subject- or image-specific irregularities. Thus, according to embodiments disclosed herein, an adaptive transmit model may select transmit lines in patterns that balance both frame rate and resolution, while selecting transmit line patterns that also are specific to an image being scanned. In some examples, the adaptive transmit model as disclosed herein may dynamically select transmits based on further constraints such as available power or data rate for devices. The adaptive transmit model may be trained using reinforcement learning techniques to configure optimal transmit line patterns to balance frame rate and image resolution in a task- and image-aware manner based on a reward structure of the reinforcement learning techniques.

Referring to FIG. 3, an image processing system 302 is shown, in accordance with an exemplary embodiment. In some embodiments, image processing system 302 is incorporated into the ultrasound imaging system 100. For example, the image processing system 302 may be provided in the ultrasound imaging system 100 as the processor 116 and memory 120. In some embodiments, at least a portion of image processing system 302 is included in a device (e.g., edge device, server, etc.) communicably coupled to the ultrasound imaging system via wired and/or wireless connections. In some embodiments, at least a portion of image processing system 302 included in a separate device (e.g., a workstation), which can receive images/maps from the ultrasound imaging system or from a storage device which stores the images/data generated by the ultrasound imaging system. Image processing system 302 may be operably/communicatively coupled to a user input device 332 and a display device 334. In one example, the user input device 332 may comprise the user interface 115 of the ultrasound imaging system 100, while the display device 334 may comprise the display device 118 of the ultrasound imaging system 100.

Image processing system 302 includes a processor 304 configured to execute machine readable instructions stored in non-transitory memory 306. Processor 304 may be single core or multi-core, and the programs executed thereon may be configured for parallel or distributed processing. In some embodiments, the processor 304 may optionally include individual components that are distributed throughout two or more devices, which may be remotely located and/or configured for coordinated processing. In some embodiments, one or more aspects of the processor 304 may be virtualized and executed by remotely-accessible networked computing devices configured in a cloud computing configuration.

Non-transitory memory 306 may store an adaptive transmit model 308, training module 310, and ultrasound image data 312. Adaptive transmit model 308 may include one or more machine learning models, such as deep learning networks, comprising a plurality of weights and biases, activation functions, loss functions, gradient descent algorithms, and instructions for implementing the one or more deep neural networks to process input ultrasound images. For example, adaptive transmit model 308 may store instructions for outputting a number and/or a pattern of transmit lines for acquiring a subsequent ultrasound image based on an input ultrasound image and a selected imaging task. Aspects of adaptive transmit model 308 (e.g., weights, biases) may be learned by reinforcement learning techniques depending on a plurality of conditions including but not limited to an imaging task, a beamformer used to generate the ultrasound image, and a desired image quality metric (e.g., resolution, contrast to noise ratio, etc.). In one example, a number and/or pattern of transmit lines for a lung imaging task may be different than a number and/or pattern of transmit lines for a liver imaging task. Adaptive transmit model 308 may include trained and/or untrained neural networks and may further include training routines, or parameters (e.g., weights and biases), associated with one or more neural network models stored therein.

Non-transitory memory 306 may further include training module 310, which comprises instructions for training adaptive transmit model 308 using reinforcement learning techniques, including an agent 309 and an environment 311. In training adaptive transmit model 308 using training module 310, a reward-based incentive may be implemented such that actions resulting in optimal outcomes are rewarded. Rewards may be generally represented as numerical values, where higher numerical values correlate to higher rewards. Agent 309 may include learning and decision-making components of training module 310, such that agent 309 may aim to take actions that maximize a reward so adaptive transmit model 308 may learn optimal actions to take based on a reward-seeking nature of agent 309. Environment 311 may include any component of training module 310 not included in agent 309, including but not limited to interactions available to agent 309, rewards, and tasks. Agent 309 may learn by taking actions that lead to reward-based outcomes, and after a plurality of interactions with environment 311, agent 309 may optimize actions taken such that rewards from environment 311 are maximized. Adaptive transmit model 308 may train using reinforcement learning in training module 310 such that adaptive transmit model 308 may recognize an optimal amount and pattern of transmit lines for an imaging task to balance frame rate and image quality. In some embodiments, the training module 310 is not included in the image processing system 302. The adaptive transmit model 308 thus includes trained and validated network(s).

Non-transitory memory 306 may further store ultrasound image data 312, such as ultrasound images captured by the ultrasound imaging system 100 of FIG. 1. The ultrasound images of the ultrasound image data 312 may be stored temporarily while the ultrasound images are used to train the adaptive transmit model 308. However, in examples where training module 310 is not disposed at the image processing system 302, the images usable for training the adaptive transmit model 308 may be stored elsewhere.

In some embodiments, the non-transitory memory 306 may include components included in two or more devices, which may be remotely located and/or configured for coordinated processing. In some embodiments, one or more aspects of the non-transitory memory 306 may include remotely-accessible networked storage devices configured in a cloud computing configuration.

User input device 332 may comprise one or more of a touchscreen, a keyboard, a mouse, a trackpad, a motion sensing camera, or other device configured to enable a user to interact with and manipulate data within image processing system 302. In one example, user input device 332 may enable a user to make a selection of an ultrasound image to use in training a machine learning model or to request that transmits for a particular ultrasound image acquisition be optimized.

Display device 334 may include one or more display devices utilizing virtually any type of technology. In some embodiments, display device 334 may comprise a computer monitor, and may display ultrasound images. Display device 334 may be combined with processor 304, non-transitory memory 306, and/or user input device 332 in a shared enclosure, or may be peripheral display devices and may comprise a monitor, touchscreen, projector, or other display device known in the art, which may enable a user to view ultrasound images produced by an ultrasound imaging system, and/or interact with various data stored in non-transitory memory 306.

It should be understood that image processing system 302 shown in FIG. 3 is for illustration, not for limitation. Another appropriate image processing system may include more, fewer, or different components.

FIG. 4 schematically shows an example reinforcement learning architecture 400 for training an adaptive transmit model using reinforcement learning. Reinforcement learning architecture 400 is a non-limiting example of training module 310 and thus may include a framework with an agent, such as agent 309, and an environment, such as environment 311. Using the components of the training module described herein, reinforcement learning architecture 400 may train the adaptive transmit model to adaptively select an optimal number of transmits for an acquisition of an ultrasound image depending on the ultrasound image being acquired (e.g., the anatomical features to be imaged, the location of the anatomical features, etc.), a task for which the acquisition is performed (e.g., the reason the image is being obtained, such as to view B-lines in a lung image or visualize a lesion), and a beamformer being used. The beamformer may refer to the configuration of the ultrasound probe (e.g., the number and arrangement of the ultrasound transducers) as well as how transmits and receives are controlled/processed (e.g., retrospective transmit beamforming, multi-line acquisition, deep learning based beamforming, etc.).

Agent 309 may include a state 402, a reinforcement learning model 404, and an action 406. Reinforcement learning model 404 is a non-limiting example of adaptive transmit model 308, and may be a partially trained or untrained version of the adaptive transmit model 308. Agent 309 may include learning and decision-making components of reinforcement learning architecture 400, such that agent 309 may aim to take actions that maximize a reward so the adaptive transmit model may learn optimal actions to take based on a reward-seeking nature of agent 309. Agent 309 may include instructions that are executable to generate lower quality images, based on output from the reinforcement learning model 404, that are also used as training data for the reinforcement learning model 404.

State 402 may include a representation of a present status for an imaging task. State 402 may be an image having an image quality metric and generated with a given number of transmits, represented by I_{M′},E_{M′}. The value I_{M′} may represent an image generated with a given number and pattern of transmits, where the value E_{M′} represents the transmit number and pattern. For example, state 402 may represent a current image acquired with a given current number of transmit lines and having a given image quality.

Reinforcement learning model 404 may be an artificial intelligence learning based model (e.g., a neural network) that is being trained via the training module 400. In a non-limiting example, the reinforcement learning model 404 may be an untrained or partially trained version of the adaptive transmit model 308 of FIG. 3. Current state 402 of agent 309 (e.g., the current image) may be input into reinforcement learning model 404. Reinforcement learning model 404 may take an action 406 based on the input. In the present case, the action is selection of a number of transmits as well as a transmit pattern.

Action 406 may include a calculated output from reinforcement learning model 404 that agent 309 may use to generate a next image that is then evaluated in an environment, such as environment 311. Action 406 may be represented as E_{K}, which may be an additional amount of transmit lines to apply to acquire a next image in the ultrasound scan. The value K may be any quantity inclusively between 1 and a total number of possible transmits for the ultrasound scan while being a quantity such that K and M′ are not the same, ensuring that a change in a transmit pattern occurs with each action. For example, reinforcement learning model 404 may calculate additional transmits to add to a current transmit pattern to attempt to increase image quality, which is evaluated by environment 311. Action 406 may include positional data relating to additional transmits such that K represents not just an additional number of transmits, but also the location of those transmits.

Environment 311 may include an instance 408 and a reward 410. Environment 311 may include any component of training module 310 not included in agent 309, including but not limited to interactions available to agent 309, rewards, and tasks. For example, environment 311 may include instructions that are executable to determine an image quality metric of a current image, compare the image quality metric of the current image to an image quality metric of a prior image, and calculate a reward based on the difference in the image quality.

Instance 408 may include an updated representation of a present status for an imaging task. Instance 408 may be an image quality metric for an image generated with a given number of transmits as a result of action 406. Instance 408 may be determined by I_{M′+K} given E_{M′+K} such that I_{M′+K} is the image quality metric of the current image obtained with M′+K transmits.

In one example, as a result of action 406 (e.g., indicating additional transmit lines), a subsequent/next image is generated, which may alter the image quality metric (e.g., image resolution). Instance 408 may update state 402 as a result of being updated by action 406, which may subsequently update action 406 in agent 309 for a future action. In other words, after the image quality metric is determined at instance 408, the current image I_{M′+K} is updated to be I_{M′} and is entered as input to the model 404.

Instance 408 may also trigger a reward 410. Reward 410 may include a consequential distribution of values depending on a condition or a plurality of conditions. Reward 410 may be distributed to agent 309, specifically to reinforcement learning model 404, so that the reinforcement model being trained may receive feedback for a calculated and implemented action. Reward 410 may reward positive values to agent 309 for actions that accomplish a goal, or lead to accomplishing a goal, for a current imaging task. Reward 410 may reward negative values to agent 309 for actions that do not accomplish a goal or regress a goal metric for a current imaging task. In one example, reward 410 may be determined by equation 1 below.

+10 if ∥I_{(M′+K)}−I_{M′}∥<ε, else −1 if M′+K>M (equation 1)

The value M represents the number of transmits used to generate the prior (or original) image and the value E may represent a threshold difference, based on the image quality metric (I_{M}), such that in this example, image quality may be compared between images before and after an action is taken, and if an absolute value of the difference between the images before and after the action is taken does not exceed the threshold, a positive reward may be given. In one example, I_{M} may be an image metric such as mean squared error (MSE), structural similarity image metric (SSIM), or contrast to noise ratio (CNR), where each of these possible image metrics have a corresponding ε. If the absolute value of the difference between the images before and after the action is taken does exceed the threshold, a negative reward is given when the total number of transmits exceeds the initial number of transmits (which may occur in almost all instances). In the example shown, the positive reward may be 10 and the negative reward may be −1, but the reward values may have different values than 10 and −1 without departing from the scope of this disclosure, such as the negative reward being smaller in absolute value than the positive reward. In one example, the reward values may be input by a user. In this way, a relatively large positive reward may be applied once a next/subsequent image has a quality that is close to the quality of the prior image, indicating that image quality has been maximized, while the negative reward applied for additional transmits may act to minimize the total number of transmits. In some examples, a negative reward (e.g., of −1) may be applied for each additional transmit that is added to the transmit pattern. In an alternate embodiment of FIG. 4, the negative reward may have a larger absolute value than the positive reward if the model is to be trained to prioritize a high frame rate in generating ultrasound images. In general, the reward values may be selected to train the model to prioritize a given parameter (e.g., image quality, low frame rate, etc.) such that the agent is positively rewarded when the task is complete and negatively rewarded whenever additional time to complete the task is taken.

By using a system of reinforcement learning as depicted in FIG. 4, when a first image is obtained with a first number of transmits, its state (e.g., the first image) may be input into the adaptive transmit model. The adaptive transmit model may calculate a subsequent quantity and pattern of transmit lines for the transmit pattern for generating a next image.

When the next image is generated, an image quality between the current image (e.g., the next image) and the previous image (e.g., the first image) is compared, such as comparing image resolutions, CNR, or another image quality metric. This process may be iteratively repeated such that each subsequent image is input into the model to determine a subsequent transmit pattern and the image quality of each subsequent image is compared to an image quality of the immediately previous image. When the image quality is compared, a consequential reward is determined. If the image quality of the current image is further from a goal metric such as resolution than the image quality of a previous image, no reward (e.g., a reward of zero) may be applied for the difference in image quality. However, a negative reward may be applied based on an increased number of transmits. In one example, a previous image may have a relatively low resolution and a current image may have a significantly higher resolution than the previous image, so no reward may be determined as a result of the increase in image quality, which indicates that image quality is still being maximized. If the image quality of the current image is relatively close to the resolution of the previous image (within the threshold of variance), a positive reward may be determined. Once the reward reaches a threshold (e.g., a positive value) or once a positive reward is applied, the cumulative reward may be used to update the model, and the process may be repeated with a new set of images.

The adaptive transmit model may be trained to seek out maximum rewards for every action it takes as a result of the reinforcement learning techniques in training. With each subsequent image that is generated, the adaptive transmit model may seek out optimal actions to maximize reward, such as calculating transmit line quantities and patterns that may maximize image resolution while minimizing the number of transmits and hence maximizing frame rate.

In order to train the model to be task-specific, the images used to train the model may all be images acquired in order to perform the task. For example, if the model is intended to select transmits for imaging the lungs to visualize B-lines, all of the training images may include images of the lungs where B-lines are visualized. If the model is intended to select transmits for imaging a valve of the heart, all of the training images may include images of the heart with the valve visible.

In some examples, the model may select transmits in a beamformer-specific manner. To accomplish this, the model may be partially trained before undergoing further training via the reinforcement learning architecture described herein, where the model may be partially trained to select K (e.g., the number/pattern of transmits) in a manner that is beamformer-specific. Additionally or alternatively, the training images used to train the model as discussed herein may all be formed using the same beamformer. Because the image quality of the images is dependent on the particular beamformer used to generate the images, training the model on beamformer-specific images where image quality is prioritized will act to train the model for the specific beamformer. In still further examples, the model may be trained to account for further constraints, such as an available amount of power to operate the ultrasound probe, available bandwidth for data transfer from the ultrasound probe, etc. To train the model to consider available power or bandwidth, additional rewards may be calculated by the environment that penalize power consumption or data amounts and/or reward lowered power consumption and/or data amounts in order to achieve a goal with fewer transmits adhering to any power consumption boundaries. Fewer transmits may result in lower power consumption, and thus a model trained to prioritize fewer transmits may be utilized when power availability is low (e.g., as determined by the battery state of charge of the ultrasound probe and/or user input).

The agent 309 has been described herein as being configured to generate images based on the output of the model 404, e.g., such that the generated images correspond to images acquired with the number/pattern of transmits output by the model. In some examples, the agent may utilize an initial training dataset that includes a plurality of training images all acquired at high image quality with a high (e.g., the maximum) number of transmits that are uniformly spaced (or have a pattern selected to optimally image for a specific task), also referred to herein as high-transmit images. When an episode of training the model commences, the agent may select a first high-transmit image and selectively remove data from the image so that a first low-transmit image is formed. The first low-transmit image may mimic an image acquired with a low number of uniformly spaced transmits, such as 10% of the transmits of the high-transmit image. Once the model outputs a new number/pattern of transmits, the agent may again selectively remove data from the high-transmit image (or alternatively add data to the first low-transmit image) to form a second low-transmit image that mimics an image acquired with the number/pattern of transmits specified by the output of the model. This process may be iteratively repeated as the model continues to output suggested transmits, until image quality is maximized and the episode ends.

In other examples, the training images may be acquired in real-time during the training. In such examples, the agent may control an ultrasound probe to acquire a plurality of images each with a different number/pattern of transmits as specified by the model.

In this way, the agent is configured to iteratively generate, based on output from the untrained version of the adaptive transmit model, a reduced-transmit image from a full-transmit image. The environment is configured to compare a first image quality of a first iteration of the reduced-transmit image to a second image quality of a second iteration of the reduced-transmit image and apply a reward based on the comparison. Further, the environment is configured to apply a first, larger reward when a difference between the first image quality and the second image quality is less than a threshold (e.g., a reward of 10), and apply a second, smaller reward when the difference is equal to or greater than the threshold (e.g., a reward of zero), and the environment is further configured to apply a third reward, smaller than the second reward, for each iteration of the reduced-transmit image or each transmit that is added by the model (e.g., a reward of −1).

FIG. 5 shows a flow chart illustrating an example method 500 for acquiring an ultrasound image with an optimized transmit pattern. Optimizing the transmit pattern may include any modification from an initial transmit pattern to balance image quality and frame rate so that a high quality image is obtained without significantly lowering frame rate in relation to an imaging task. Method 500 is described with regard to the systems and components of FIGS. 1, 3, and 4, though it should be appreciated that the method 500 may be implemented with other systems and components without departing from the scope of the present disclosure. Method 500 may be carried out according to instructions stored in non-transitory memory of a computing device, such as image processing system 302 of FIG. 3.

At 502, ultrasound images are acquired and displayed on a display device. For example, the ultrasound images may be acquired with the ultrasound probe 106 of FIG. 1 and displayed to an operator via display device 118. The images may be acquired and displayed in real time or near real time, and may be acquired with default or user-specified scan parameters (e.g., default depth, frequency, default transmit pattern, etc.).

At 504, method 500 determines if a request to optimize transmits is received. The request may be automatic based on predetermined settings for a current ultrasound scan, or the request may be a manual input by an operator. If the request to optimize transmits is not received, method 500 may continue to 502 to acquire and display more ultrasound images.

If the request to optimize transmits is received, method 500 may continue to 506, which includes controlling an ultrasound probe to acquire a sparse transmit ultrasound image. An initial predetermined transmit pattern may be used to acquire the sparse transmit ultrasound image regardless of imaging task. In one example, a transmit pattern for an initial lung scan may also be the same pattern used for an initial liver scan. The initial predetermined transmit pattern may include a limited number of transmits, such as 10% of a total possible number of transmits. The initial predetermined transmit pattern may include the transmits being evenly spaced apart.

At 508, method 500 includes entering the sparse transmit ultrasound image and a current task as input to an adaptive transmit model. The adaptive transmit model may be selected based on the imaging task (e.g., an adaptive transmit model that is specific to B-line imaging may be selected when the current task is B-line imaging). The adaptive transmit model may be trained according to reinforcement learning techniques described with respect to FIG. 4 to calculate a quantity and pattern of transmit lines for a next transmit pattern when a next image may be acquired during the ultrasound scan. The current task may be received via user input (e.g., the user may select the task from a menu or otherwise enter an input that identifies the task) or the current task may be determined automatically or semi-automatically based on a selected imaging protocol. For example, at the start of the imaging session, the user may specify the type of exam being conducted (e.g., abdominal scan, lung scan, echocardiogram) and the current task may be determined based on the type of exam, current progress through the exam, current anatomy being imaged, etc. As explained previously, the current task may be the reason the images are being obtained, such as a specific diagnostic goal and/or specific anatomical features to be imaged.

At 512, method 500 includes receiving a transmit pattern as output from the adaptive transmit model. The transmit pattern output from the adaptive transmit model may be different from the transmit pattern used to acquire the ultrasound image entered into the model at 508. The transmit pattern output may differ in quantity of transmit lines and/or placement of transmit lines as a result of calculations performed by the adaptive transmit model.

At 514, method 500 includes controlling an ultrasound probe to acquire an ultrasound image or a plurality of ultrasound images with the transmit pattern output from the adaptive transmit model. Once the adaptive transmit model has output the transmit pattern, each subsequent image may be acquired with the specified transmit pattern until imaging ends or the user requests a new transmit pattern be identified. Method 500 then ends. In this way, a number of transmit lines and/or a pattern of transmit lines for acquiring an ultrasound image may be dynamically updated based on a prior ultrasound image and a task to be performed with the ultrasound image, which allows for optimal transmit numbers/patterns to be selected and used for image acquisition in a subject-, ultrasound operator-, and imaging task-specific manner. By doing so, imaging frame rate may be increased to reduce motion related artifacts while minimizing image quality reductions.

Turning now to FIG. 6, a method 600 for training an adaptive transmit model for an imaging task using reinforcement learning techniques is presented. Method 600 may be carried out according to instructions stored in non-transitory memory of a computing device, such as image processing system 302 of FIG. 3 (or on a separate computing device, when training of the adaptive transmit model is carried out on a different computing device).

At 602, method 600 includes receiving an indication of a task for the training model. Tasks may be chosen by a training module, such as training module 310 of FIG. 3, or a user (e.g., an expert clinician). In one example, a task may be scanning B-lines in a pair of lungs.

At 606, method 600 includes generating a prior image. A predetermined initial transmit pattern may be used to generate the prior image. The prior image may be generated from a selected high-transmit image of a dataset of high-transmit images (e.g., training images) that may be used as a source material to generate a purposefully lower resolution or sparse transmit image using the initial transmit pattern. As an example, if the selected high-transmit image was acquired with 140 transmit lines, the prior image may be generated to mimic an image acquired with 14 transmit lines.

At 608, method 600 includes entering the prior image into the untrained adaptive transmit model and receiving an action (e.g., updated transmit pattern) from the adaptive transmit model. The updated transmit pattern may include additional transmit lines and positional information for the additional transmit lines.

At 612, method 600 includes performing the action by generating a next image with the updated transmit pattern. The next image may be generated from the same original high transmit image from the dataset of high transmit images used to generate the prior image, but the updated transmit pattern may be used instead of the initial transmit pattern. The updated transmit pattern may include the initial transmit pattern and the additional transmits output by the model.

At 614, method 600 includes calculating a reward based on an image quality difference between the prior image and the next image. Image quality comparisons may be performed by comparing resolutions or other quality metrics (e.g., contrast to noise ratio, image brightness, and/or region of interest visibility) between the prior image and the next image. A positive reward may be applied to the adaptive transmit model once the next image has an image quality that is close to the image quality of the prior image (e.g., less than a specified error, such as the threshold of difference explained above with respect to FIG. 4), indicating that image quality has been maximized. Each time additional transmits are added, image quality may increase. However, the increases in image quality may decrease and finally plateau once an optimized transmit pattern is identified, and additional transmits may not further improve image quality. A negative reward may be applied to the adaptive transmit model to minimize the total number of transmits. For example, a negative reward may be applied when the total number of transmits used to form the next image is greater than the initial number of transmits (e.g., used to form the first image, which in this example is the prior image). In one example, a value of 10 may be used as the positive reward, and a value of −1 may be used as the negative reward.

At 616, method 600 includes updating the action in the agent by entering the next image into the adaptive transmit model. The updated action may include a further updated transmit pattern to generate a new ultrasound image.

At 618, method 600 includes updating the state in the agent by performing the updated action, which includes generated a further next ultrasound image (e.g., the new ultrasound image) with the further updated transmit pattern.

At 620, method 600 includes repeating the reward calculations, action updates, and state updates until an end goal is reached. The end goal may include the positive reward being applied, due to the image quality being maximized, or another suitable reward being applied, such as the reward reaching a threshold.

At 622, method 600 includes updating the adaptive transmit model based on the reward. The reward may be cumulative over the episode, such that if the adaptive transmit model needed 10 outputs to maximize the image quality, the reward that is applied may be 9 (e.g., 10 for maximizing the image quality but −1 for the additional model outputs required to reach the positive reward).

This process may be repeated until the adaptive transmit model is able to identify, for each new low-transmit image, the transmit pattern that will maximize image quality without using any additional transmits beyond the point at which the image quality is maximized. Each low-transmit image is generated from a different high-transmit image. Thus, once the positive reward is applied for a given low-transmit image set (generated from a high-transmit image), a new high-transmit image is selected and a new low-transmit image is formed from the new high-transmit image and used as the prior image. Additional low-transmit images are then formed based on the output of the adaptive transmit model until the positive reward is applied, and the reward is used to update the model. As the model learns the optimal transmit pattern, the number of outputs from the model to maximize the image quality will decrease until a point is reached where it may be determined that the model is trained.

Thus, using the reinforcement learning techniques, the adaptive transmit model may try to get the positive reward with a fewest number of attempts. As the adaptive transmit model is trained, it may calculate transmit patterns from a sparse transmit pattern ultrasound image to generate an ultrasound image satisfying a goal for an imaging task in a minimal amount of image generations with minimal to no user involvement during the training, reducing overall times and a usage of computational resources for ultrasound scans. Method 600 then ends.

FIGS. 7A-7C show a series of transmit patterns for forming ultrasound images during training of the adaptive transmit model, representing an adaptive transmit sampling process. Adaptive transmit sampling may be used in the methods in FIGS. 5-6 by an adaptive transmit model trained by reinforcement learning techniques used in FIG. 4.

FIG. 7A shows an initial sparse transmit pattern in which a uniform pattern of transmit lines may be transmitted to generate an ultrasound image. The initial sparse transmit pattern may include uniformly spaced transmits, as the initial sparse transmit pattern may not favor a specific region of interest.

FIG. 7B shows a transmit pattern on an ultrasound image with additional lines applied by the adaptive transmit model relative to FIG. 7A. In one example, FIG. 7B may reflect a transmit pattern output by the adaptive transmit model using the ultrasound image of FIG. 7A as input. Additional lines generated in this transmit pattern are shown as dashed lines and may be positioned in regions of interest to increase image resolution and contrast based on the input image and imaging task. However, the transmit pattern shown in FIG. 7B may not result in maximized image quality, and thus the adaptive transmit model may continue to output transmit patterns based on new input images (e.g., the image formed from the transmit pattern shown in FIG. 7B may be input to the adaptive transmit model on a next iteration).

FIG. 7C shows a transmit pattern on an ultrasound image with additional lines applied by the adaptive transmit model relative to FIG. 7B. In one example, FIG. 7C may reflect a final transmit pattern output by the adaptive transmit model using the ultrasound image in FIG. 7B as input, satisfying image quality goals (e.g., maximized image quality) and the imaging task. As appreciated from FIG. 7C, the additional lines generated in this transmit pattern may be non-uniformly spaced such that the transmits are spaced closer together/have a higher density in regions of interest specific to the image and the imaging task. For example, the transmits are positioned such that the transmits are preferentially transmitted to anatomical regions of interest (e.g., the lungs) and are not transmitted to areas that lack anatomy/anatomy of interest.

A technical effect of dynamically selecting transmit line patterns during ultrasound scans based on an image and an imaging task, such as scanning for B-lines in a pair of lungs, is that image quality may be maximized without unduly lowering frame rate by targeting the transmit lines to anatomical regions of interest as identified in the image and specified by the imaging task. Ultrasound images may be acquired in a fast manner using adaptive transmit patterns. Another technical effect of the adaptive transmit model is that initial transmit patterns may be standardized across a plurality of ultrasound scanning regions, which may decrease a number of scans to get a goal frame rate and resolution for an ultrasound image.

In one embodiment, a method comprises dynamically updating a number of transmit lines and/or a pattern of transmit lines for acquiring an ultrasound image based on a prior ultrasound image and a task to be performed with the ultrasound image, and acquiring the ultrasound image with an ultrasound probe controlled to operate with the updated number of transmit lines and/or the updated pattern of transmit lines.

In a first example of the method, the method further comprises dynamically updating the number of transmit lines and/or pattern of transmit lines for acquiring the ultrasound image, acquiring the prior ultrasound image with a first number of transmit lines and a first pattern of transmit lines, and entering the prior ultrasound image to an adaptive transmit model configured to output the updated number of transmit lines and/or the updated pattern of transmit lines based on the prior ultrasound image and the task. In a second example of the method, optionally including the first example, the first number of transmit lines is smaller than the updated number of transmit lines. In a third example of the method, optionally including one or both of the first and second examples, the first pattern of transmit lines includes the transmit lines being uniformly spaced apart and the updated pattern of transmit lines includes at least some of the transmit lines being non-uniformly spaced apart. In a fourth example of the method, optionally including one or more or each of the first through third examples, the adaptive transmit model is one of a plurality of adaptive transmit models and the adaptive transmit model is selected from among the plurality of adaptive transmit models based on the task. In a fifth example of the method, optionally including one or more or each of the first through fourth examples, the adaptive transmit model is trained using reinforcement learning. In a sixth example of the method, optionally including one or more or each of the first through fifth examples, training the adaptive transmit model comprises: entering an initial image to an untrained version of the adaptive transmit model, the initial image generated with a first number transmit lines, receiving, as an output from the untrained version of the adaptive transmit model, one or more additional transmit lines to include with the first number of transmit lines, thereby forming a second number of transmit lines, generating a subsequent image with the second number transmit lines, comparing a quality of the initial image to a quality of the subsequent image and calculating a reward based on the comparison, and updating the untrained version of the adaptive transmit model based on the reward. In a seventh example of the method, optionally including one or more or each of the first through sixth examples, the task to be performed includes one or more of an anatomical feature to be imaged in the ultrasound image and a diagnostic goal of the ultrasound image.

In another embodiment, a system comprises a memory storing instructions, and a processor communicably coupled to the memory and when executing the instructions, configured to control an ultrasound probe to acquire a first image of a subject with a first number of transmit lines, enter the first image as input to an adaptive transmit model trained to output a second number of transmit lines based on the first image, and control the ultrasound probe to acquire a second image of the subject with the second number of transmit lines, the second number of transmit lines larger than the first number of transmit lines.

In a first example of the system, the adaptive transmit model is selected from a plurality of adaptive transmit models based on a task to be performed with second image. In a second example of the system, optionally including the first example, the adaptive transmit model is selected from a plurality of adaptive transmit models based on a type of beamformer used to generate the second image. In a third example of the system, optionally including one or both of the first and second examples, the adaptive transmit model is trained using a reinforcement learning architecture that comprises an agent and an environment, the agent including an untrained version of the adaptive transmit model. In a fourth example of the system, optionally including one or more or each of the first through third examples, the agent is configured to iteratively generate, based on output from the untrained version of the adaptive transmit model, a reduced-transmit image from a full-transmit image. In a fifth example of the system, optionally including one or more or each of the first through fourth examples, the environment is configured to compare a first image quality of a first iteration of the reduced-transmit image to a second image quality of a second iteration of the reduced-transmit image and apply a reward based on the comparison. In a sixth example of the system, optionally including one or more or each of the first through fifth examples, the environment is configured to apply a first, larger reward when a difference between the first image quality and the second image quality is less than a threshold, and apply a second, smaller reward when the difference is equal to or greater than the threshold, and the environment is further configured to apply a third reward, smaller than the second reward, for each iteration of the reduced-transmit image.

In yet another embodiment, a method comprises responsiveness to a request to optimize transmits for acquiring an ultrasound image of a subject, acquiring a sparse transmit ultrasound image of the subject with an initial transmit pattern, entering the sparse transmit ultrasound image and a selected imaging task as inputs to an adaptive transmit model trained to output a dynamic transmit pattern based on the sparse transmit ultrasound image and the imaging task, and acquiring the ultrasound image of the subject with the dynamic transmit pattern.

In a first example of the method, acquiring the sparse transmit ultrasound image of the subject with the initial transmit pattern comprises acquiring the sparse transmit ultrasound image of the subject with a first number of transmit lines uniformly spaced apart, and wherein acquiring the ultrasound image of the subject with the dynamic transmit pattern comprises acquiring the ultrasound image of the subject with a larger, second number of transmit lines at least some of which are non-uniformly spaced apart. In a second example of the method, optionally including the first example, the ultrasound image is acquired with an ultrasound probe, and wherein the second number of transmit lines is smaller than a maximum number of transmit lines the ultrasound probe is capable of transmitting. In a third example of the method, optionally including one or both of the first and second examples, the adaptive transmit model is trained using reinforcement learning. In a fourth example of the method, optionally including one or more or each of the first through third examples, training the adaptive transmit model comprises: entering an initial image to an untrained version of the adaptive transmit model, the initial image generated with a first number transmit lines, receiving, as an output from the untrained version of the adaptive transmit model, one or more additional transmit lines to include with the first number of transmit lines, thereby forming a second number of transmit lines, generating a subsequent image with the second number transmit lines, comparing a quality of the initial image to a quality of the subsequent image and calculating a reward based on the comparison, and updating the untrained version of the adaptive transmit model based on the reward.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
dynamically updating a number of transmit lines and/or a pattern of transmit lines for acquiring an ultrasound image based on a prior ultrasound image and a task to be performed with the ultrasound image; and
acquiring the ultrasound image with an ultrasound probe controlled to operate with the updated number of transmit lines and/or the updated pattern of transmit lines.

2. The method of claim 1, wherein dynamically updating the number of transmit lines and/or pattern of transmit lines for acquiring the ultrasound image comprises:
acquiring the prior ultrasound image with a first number of transmit lines and a first pattern of transmit lines; and
entering the prior ultrasound image to an adaptive transmit model configured to output the updated number of transmit lines and/or the updated pattern of transmit lines based on the prior ultrasound image and the task.

3. The method of claim 2, wherein the first number of transmit lines is smaller than the updated number of transmit lines.

4. The method of claim 2, wherein the first pattern of transmit lines includes the transmit lines being uniformly spaced apart and the updated pattern of transmit lines includes at least some of the transmit lines being non-uniformly spaced apart.

5. The method of claim 2, wherein the adaptive transmit model is one of a plurality of adaptive transmit models and the adaptive transmit model is selected from among the plurality of adaptive transmit models based on the task.

6. The method of claim 2, wherein the adaptive transmit model is trained using reinforcement learning.

7. The method of claim 6, wherein training the adaptive transmit model comprises:
entering an initial image to an untrained version of the adaptive transmit model, the initial image generated with a first number transmit lines;
receiving, as an output from the untrained version of the adaptive transmit model, one or more additional transmit lines to include with the first number of transmit lines, thereby forming a second number of transmit lines;
generating a subsequent image with the second number transmit lines;
comparing a quality of the initial image to a quality of the subsequent image and calculating a reward based on the comparison; and
updating the untrained version of the adaptive transmit model based on the reward.

8. The method of claim 1, wherein the task to be performed includes one or more of an anatomical feature to be imaged in the ultrasound image and a diagnostic goal of the ultrasound image.

9. A system, comprising:
a memory storing instructions; and
a processor communicably coupled to the memory and when executing the instructions, configured to:
control an ultrasound probe to acquire a first image of a subject with a first number of transmit lines;
enter the first image as input to an adaptive transmit model trained to output a second number of transmit lines based on the first image; and
control the ultrasound probe to acquire a second image of the subject with the second number of transmit lines, the second number of transmit lines larger than the first number of transmit lines.

10. The system of claim 9, wherein the adaptive transmit model is selected from a plurality of adaptive transmit models based on a task to be performed with second image.

11. The system of claim 9, wherein the adaptive transmit model is selected from a plurality of adaptive transmit models based on a type of beamformer used to generate the second image.

12. The system of claim 9, wherein the adaptive transmit model is trained using a reinforcement learning architecture that comprises an agent and an environment, the agent including an untrained version of the adaptive transmit model.

13. The system of claim 12, wherein the agent is configured to iteratively generate, based on output from the untrained version of the adaptive transmit model, a reduced-transmit image from a full-transmit image.

14. The system of claim 13, wherein the environment is configured to compare a first image quality of a first iteration of the reduced-transmit image to a second image quality of a second iteration of the reduced-transmit image and apply a reward based on the comparison.

15. The system of claim 14, wherein the environment is configured to apply a first, larger reward when a difference between the first image quality and the second image quality is less than a threshold, and apply a second, smaller reward when the difference is equal to or greater than the threshold, and the environment is further configured to apply a third reward, smaller than the second reward, for each iteration of the reduced-transmit image.

16. A method, comprising:
responsive to a request to optimize transmits for acquiring an ultrasound image of a subject, acquiring a sparse transmit ultrasound image of the subject with an initial transmit pattern;
entering the sparse transmit ultrasound image and a selected imaging task as inputs to an adaptive transmit model trained to output a dynamic transmit pattern based on the sparse transmit ultrasound image and the imaging task; and
acquiring the ultrasound image of the subject with the dynamic transmit pattern.

17. The method of claim 16, wherein acquiring the sparse transmit ultrasound image of the subject with the initial transmit pattern comprises acquiring the sparse transmit ultrasound image of the subject with a first number of transmit lines uniformly spaced apart, and wherein acquiring the ultrasound image of the subject with the dynamic transmit pattern comprises acquiring the ultrasound image of the subject with a larger, second number of transmit lines at least some of which are non-uniformly spaced apart.

18. The method of claim 17, wherein the ultrasound image is acquired with an ultrasound probe, and wherein the second number of transmit lines is smaller than a maximum number of transmit lines the ultrasound probe is capable of transmitting.

19. The method of claim 16, wherein the adaptive transmit model is trained using reinforcement learning.

20. The method of claim 19, wherein training the adaptive transmit model comprises:
entering an initial image to an untrained version of the adaptive transmit model, the initial image generated with a first number transmit lines;
receiving, as an output from the untrained version of the adaptive transmit model, one or more additional transmit lines to include with the first number of transmit lines, thereby forming a second number of transmit lines;
generating a subsequent image with the second number transmit lines;
comparing a quality of the initial image to a quality of the subsequent image and calculating a reward based on the comparison; and
updating the untrained version of the adaptive transmit model based on the reward.
